SEQUENCE LISTING (21 sequences)

SEQ ID NO: 1 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 2 - protein, 290 aa, Homo sapiens
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 3 - protein, 236 aa, Homo sapiens
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 4 - DNA, 180 bp, Homo sapiens
tccttcacaatttctgcagccactccgtggccaccgtcaccttattcctgactgccacaagagtctttcaatattcctttgattgcctattccttctgaaatctaccttttcctctaatagggcaattcatcattttcaaatgcaatttttactctgatctagaacttactgtgaatcct

SEQ ID NO: 5 - DNA, 180 bp, Homo sapiens
tccttcacaatttctgcagccactctgtggcgaccgtcagcttattcctgaaggccacaagagtctttcaatattcctttgattgcctattccttctgaaatctaccttttcctctaatagagcaactcatcattttcaaatgcaatttttactctgatctagaacttactgtgaatcct

SEQ ID NO: 6 - DNA, 179 bp, Homo sapiens
tgagataggtaatattccattttacagatgaagtaaccgaggtgcaaaaataaataaataagtttccctaaggtcacatcaaagacttcaaagcctgtatatttaaccagtaagtaaaagatttgaacaagcactaatatcctatgatcccattaagtcatccacaaaacatctctagg

SEQ ID NO: 7 - DNA, 180 bp, Homo sapiens
tgagataggtaatattccattttacagatgaagtaaccgaggtgcaaaaataaataaataagttgcccctaaggtcacatcaaagacttcaaagcctgtatatttaaccagtaagtaaaagatttgaagaagcactaatatcctatgatcccattaagtcatccacaaaacatctctagg

SEQ ID NO: 8 - DNA, 178 bp, Homo sapiens
ggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttct

SEQ ID NO: 9 - DNA, 179 bp, Homo sapiens
ggactgaggattggggtgggggtggggtggaaaagaaagtacagaacaaaccctgtgtcactgtctcaagttaagctaagtgaacagaactatctcagcatcagaatgagaaagcctgagaagaaagaaccaaccagaagcacacaggaaggaaagcgcaggaggtgaaaatgctttct

SEQ ID NO: 10 - DNA, 22 bp, Homo sapiens
accgtcaccttattcctgactg

SEQ ID NO: 11 - DNA, 26 bp, Homo sapiens
ctgagatagttctgttcacttagcaa

SEQ ID NO: 12 - DNA, 27 bp, Homo sapiens
aggcaggaagtattttcatcataattc

SEQ ID NO: 13 - DNA, 22 bp, Homo sapiens
aacttcccagtgtgattgcagg

SEQ ID NO: 14 - DNA, 19 bp, Homo sapiens
gacacatttttactcccaa

SEQ ID NO: 15 - DNA, 19 bp, Homo sapiens
ttgatgtgaccttagggaa

SEQ ID NO: 16 - DNA, 26 bp, Homo sapiens
gtcacatatttacagaatggcaaagg

SEQ ID NO: 17 - DNA, 24 bp, Homo sapiens
ccaactcaacttcccagtgtgatt

SEQ ID NO: 18 - DNA, 18 bp, Homo sapiens
aggcaggaagtattttca

SEQ ID NO: 19 - DNA, 26 bp, Homo sapiens
aaagccccctgcagaagtaggagccg

SEQ ID NO: 20 - DNA, 26 bp, Homo sapiens
caagccccctgcagaagtaggagccg

SEQ ID NO: 21 - DNA, 10 bp, Homo sapiens
tcatgcctgc

(19) United States
(12) Patent Application Publication
(10) Pub. No.: US 2009/0142763 A1
(43) Pub. Date: Jun. 4, 2009

(54) NESTED PCR-BASED METHOD FOR SPECIFIC GENOTYPING OF THE FC GAMMA RECEPTOR IIIA GENE

(75) Inventors: Michael E. BURCZYNSKI, Collegeville, PA (US); Jennifer A. Isler, Brookfield, CT (US); Anna M. Slager, Lansdale, PA (US); Wenyan Zhong, Collegeville, PA (US)

Correspondence Address: FITZPATRICK CELLA (WYETH), 30 ROCKEFELLER PLAZA, NEW YORK, NY, 10112-3800, US

(73) Assignee: Wyeth, Madison, NJ (US)

(21) Appl. No.: 12/196,542

(22) Filed: Aug. 22, 2008

Related U.S. Application Data
(60) Provisional application No. 60/957,185, filed on Aug. 22, 2007.

(51) Int. Cl. C12Q 1/68

(52) U.S. Cl. 435/6

(57) ABSTRACT

The application describes a novel method for detection of a single nucleotide polymorphism, e.g., the 158 F/V polymorphism, in the FcγRIIIa gene using nested PCR to amplify large gene amplicons to aid in, e.g., genotyping applications. Thus, based on the results obtained in FcγRIIIa genotyping analysis, the present invention provides specific, efficient, reproducible, and accurate detection of such polymorphisms in genomic DNA.

### CROSS-REFERENCE TO RELATED APPLICATION

This application claims the benefit of U.S. Provisional Application No. 60/957,185, filed Aug. 22, 2007, which is incorporated herein by reference in its entirety.

### BACKGROUND OF THE INVENTION

1. Field of the Invention

The present invention relates to a novel method of PCR amplification, specifically a method of amplifying large gene amplicons to aid in, e.g., genotyping applications. More specifically, the invention relates to amplifying an Fc-γ receptor IIIa (FcγRIIIa)-specific amplicon to allow for, e.g., specific genotyping of the FcγRIIIa gene, in order to facilitate, e.g., identification of polymorphisms, e.g., clinically relevant polymorphisms, e.g., the FcγRIIIa 158 F/V polymorphism.

2. Relevant Background Art

Cells often signal an infection by expressing, on their surface, foreign proteins that are recognized by antibodies. In a process called antibody-dependent cell-mediated cytotoxicity (ADCC), Fc receptors on the surface of cytotoxic cells, e.g., natural killer cells (NK cells), recognize the antibody-coated infected cell, which step subsequently leads to cell destruction.

The FcγRIIIa gene, also known as CD16 gene, is one of several Fc receptor genes; it encodes the FcγRIIIa receptor, which is expressed on the surface of natural killer cells, monocytes and macrophages. Interactions of natural killer cells with IgG antibodies via FcγRIIIa induce signal transduction and lead to ADCC as well as release of various cytokines. The FcγRIIIa gene displays a functional polymorphism, referred to as FcγRIIIa 158 F/V, in which a T to G nucleotide substitution at position 101,411 (GenBank Accession No. AL590385; T is present on antisense strand) results in a phenylalanine to valine amino acid substitution at amino acid residue 158 of the mature protein, or position 176 of unprocessed protein, or position 212 in GenBank Accession No. NP_{—}000560.5 (GI:50726979), as shown in Table 1 and FIG. 1. The polymorphism alters receptor function by increasing its affinity for immunoglobulin G1 (IgG1), thereby increasing the level of natural killer cell activation after FcγRIIIa engagement. Both FcγRIIIa alleles are well represented in Caucasian and African-American populations, although the FcγRIIIa 158F allele appears to be more prevalent, as shown in Table 2.

TABLE 1
FcγRIIIa 158 F/V Polymorphism

| Allele | Codon^{a} | Amino Acid |
|---|---|---|
| 158F | TTT | Phenylalanine |
| 158V | GTT | Valine |

^{a}The polymorphic nucleotide is bold faced and underlined.

TABLE 2
FcγRIIIa Genotype Frequencies in Healthy Subjects

| | | African-American |
|---|---|---|
| Genotype | Caucasian (n = 181) | (n = 152) |
| 158 F/F | 50% | 42% |
| 158 F/V | 39% | 50% |
| 158 V/V | 11% | 8% |

Table adopted from Lehrnbecher et al. (1999) Blood 94: 4220-32.

Genetic links between the low-affinity allele of FcγRIIIa (158F) and autoimmune diseases such as systemic lupus erythematosus (SLE) have been described (Wu et al. (1997) J. Clin. Invest. 100:1059-70). Several studies also suggested association between 158 F/V polymorphism and susceptibility to rheumatoid arthritis (Nieto et al. (2000) Arthritis Rheum. 43:735-39; Chen et al. (2006) Clin. Exp. Immunol. 144:10-16; Morgan et al. (2000) Arthritis Rheum. 43:2328-34). Moreover, the efficacy of rituximab, an anti-CD20 antibody used to treat some autoimmune diseases as well as B cell lymphomas, varies with respect to the 158 F/V polymorphism; individuals homozygous for the high-affinity FcγRIIIa allele (158V) typically respond to rituximab with increased success as compared to homozygotes for the low-affinity allele (158F) (Cartron et al. (2002) Blood 99:754-58; Anolik et al. (2003) Arthritis Rheum. 48:455-59). Other studies on associations between the FcγRIIIa 158 F/V polymorphism and disease are reviewed in van Sorge et al. (2002) Tissue Antigens 61:189-202.

The correlation between 158 F/V polymorphism and several diseases as well as disease therapies suggests the need for a high-throughput, reproducible genotyping assay for detection of the polymorphic allele. Several studies have attempted to develop such a genotyping assay (e.g., Dall'Ozzo et al. (2003) J. Immunol. Methods 277:185-92; Lee et al. (2002) Rheumatol. Int. 21:222-26; Carlsson et al. (1998) Blood 92:1526-31; Magnusson et al. (2004) Genes Immunity 5:130-37); however, none describe a strategy that results in amplification of an FcγRIIIa amplicon larger than 1700 base pairs, and therefore, none enable genotyping of a larger region of the gene. Moreover, the assay design is complicated by the close homology between FcγRIIIa and FcγRIIIb gene (i.e., 97% sequence identity) because the assay must allow specific gene amplification and genotyping.

Thus, there exists a need for a high-throughput assay that allows specific genotyping of large regions of FcγRIIIa gene (particularly, e.g., FcγRIIIa 158 F/V genotyping), as well as other genes associated with genetic diseases and/or differential responses to therapies. Such methods can aid in disease diagnosis, disease risk assessment, and design of individualized treatment.

### SUMMARY OF THE INVENTION

In at least one embodiment, the present invention provides a method of genotyping at least one polymorphism in a gene of interest, the method comprising: amplifying the gene of interest in a nested PCR reaction with gene-specific primers to generate a gene of interest-specific amplicon containing at least one polymorphic site; and performing a genotyping reaction to identify a nucleic acid at the at least one polymorphic site. In one embodiment, the downstream genotyping reaction is selected from the group consisting of pyrosequencing reaction, DNA sequencing reaction, MassARRAY MALDI-TOF, RFLP, allele-specific PCR, real-time allelic discrimination, and microarray.

In a further embodiment, the step of performing a genotyping reaction comprises: amplifying the gene of interest-specific amplicon in a second round of PCR with second-round gene-specific primers, wherein the amplification results in a biotinylated amplicon, and wherein the biotinylated amplicon comprises one biotinylated strand; purifying the biotinylated amplicon; separating the biotinylated strand of the biotinylated amplicon from the nonbiotinylated strand of the biotinylated amplicon; determining the sequence of the biotinylated strand of the biotinylated amplicon in a pyrosequencing reaction; and comparing the sequence of the biotinylated strand of the biotinylated amplicon to the known sequence of the gene of interest. In some embodiments, the gene of interest is FcγRIIIa; the at least one polymorphism is the FcγRIIIa 158 F/V polymorphism; and the size of the gene of interest-specific amplicon is greater than about 1700 base pairs (e.g., about 3234 base pairs).

In at least some further embodiments, the present invention provides the aforementioned method of genotyping at least one polymorphism in a gene of interest, wherein the gene-specific primers are 4587F and 7820R; wherein the biotinylated strand is a sense strand or an antisense strand; wherein the step of purifying the biotinylated amplicon comprises immobilization of the biotinylated amplicon on streptavidin-coated beads; and wherein the gene-specific primers will anneal to FcγRIIIa but not FcγRIIIb.

In at least some further embodiments, the present invention provides the aforementioned method of genotyping at least one polymorphism in a gene of interest, wherein the at least one polymorphic site is selected from the group consisting of polymorphisms identified in the NCBI Single Nucleotide Polymorphism database by SNP_ID NOs: 1042223, 1042222, 104222, 375794, 445509, 378618, 448312, 1042215, 1042214, 2499445, 3181668, 7539036, 1042209, 1126552, 1042207, 1042206, 17853189, 10919555, 10800579, 10800580, 10800581, 4657062, 397429, 426615, 10533383, 10624618, 36091086, 449463, 4657063, 370077, 371849, 424288, 3835614, 394678, 449443, 396716, 443082, 5778214, and 396991.

In at least one embodiment, the present invention provides a method of assessing whether a subject has, or is at risk for, a polymorphic disease comprising detecting at least one polymorphism according to the aforementioned method of genotyping.

In at least one further embodiment, the present invention provides a method of genotyping the FcγRIIIa 158 F/V polymorphism, the method comprising: amplifying FcγRIIIa in a PCR reaction with 4587F and 7820R primers to generate an FcγRIIIa-specific amplicon containing a 158 F/V polymorphic site; and performing a genotyping reaction to identify a nucleic acid at the 158 F/V polymorphic site on each allele.

### BRIEF DESCRIPTION OF THE DRAWINGS

FIG. 1 shows an alignment of unprocessed FcγRIIIa (CD16) protein sequence (SEQ ID NO: 1), FcγRIIIa protein sequence annotated in GenBank with Accession No. NP_{—}000560.5 (SEQ ID NO:2), and mature FcγRIIIa protein sequence (SEQ ID NO:3). Phenylalanine at the polymorphic site of each isoform is bolded and underlined.

FIG. 2 is a schematic representation of the primers used for the first and second rounds of PCR for both DNA sequencing and pyrosequencing analyses. CD16aPyroFB is a biotin-tagged primer. PCR primers are depicted by arrows indicating the 5′ to 3′ directionality. The hatched lines at the left of the figure indicate that the 5′ segment of the gene is not drawn to scale.

FIG. 3 is an alignment of the FcγRIIIa (SEQ ID NOs:4, 6, and 8) and FcγRIIIb (SEQ ID NOs:5, 7, and 9) genes, demonstrating that the PCR primers were designed to anneal to regions of least identity. The 4587F primer (shown in panel A) was used in the first round of PCR for both the pyrosequencing and DNA sequencing analyses. The 7820R primer (panel C) was used for the pyrosequencing analysis, and the 6014R primer (panel B) was used for the DNA sequencing analysis. CD16a and CD16b represent FcγRIIIa and FcγRIIIb, respectively. Primer sequences are shown (underlined), and 5′ to 3′ directionality is of each indicated by arrows.

FIG. 4A and FIG. 4B are representative FcγRIIIa 158 F/V pyrosequencing results. The theoretical (FIG. 4A) and actual (FIG. 4B) results (i.e., pyrograms) for selected samples of each possible genotype are shown.

FIG. 5 shows a representative FcγRIIIa 158 F/V DNA sequencing result for each possible genotype.

### DETAILED DESCRIPTION OF THE INVENTION

The present invention provides a specific, high-throughput method for identifying gene polymorphisms, e.g., FcγRIIIa gene polymorphisms, e.g., FcγRIIIa 158 F/V gene polymorphism, wherein the method comprises: (a) amplifying FcγRIIIa in a nested PCR reaction with gene-specific primers to generate an FcγRIIIa-specific amplicon containing, e.g., the 158 F/V polymorphic site; and (b) performing a genotyping reaction to identify a nucleic acid, e.g., at the 158 F/V polymorphic site on each allele.

As used herein, “pyrosequencing analysis” refers to the steps of nucleic acid manipulation and sequence analysis, e.g., genotyping, etc., that, as one of the steps, uses a pyrosequencing reaction(s). In a preferred embodiment of the invention, the pyrosequencing analysis comprises the steps of: (a) amplifying a gene of interest in a nested PCR reaction with gene-specific primers to generate a gene of interest-specific amplicon; (b) amplifying the gene of interest-specific amplicon in a second round of PCR with second-round gene-specific primers, wherein the amplification results in a biotinylated amplicon, and wherein the biotinylated amplicon comprises a biotinylated strand and a nonbiotinylated strand; (c) purifying the biotinylated amplicon; (d) separating the biotinylated strand of the biotinylated amplicon from the nonbiotinylated strand of the biotinylated amplicon; (e) determining the sequence of the biotinylated strand of the biotinylated amplicon in a pyrosequencing reaction; and (f) comparing the sequence of the biotinylated strand of the biotinylated amplicon to the known sequence of the gene of interest.

As used herein, “DNA sequencing analysis” refers to the steps of nucleic acid manipulation and sequence analysis, e.g., genotyping, pyrosequencing validation, etc., that, as one of the steps, uses a DNA sequencing reaction(s). In a preferred embodiment of the invention, the DNA sequencing analysis comprises the steps of: (a) amplifying a gene of interest in a nested PCR reaction with gene-specific primers to generate a gene of interest-specific amplicon; (b) amplifying the gene of interest-specific amplicon in a second round of PCR; and (c) sequencing the PCR product from step (b) in a DNA sequencing reaction.

Polymerase chain reaction (PCR) is a method for rapid nucleic acid amplification that is well known in the art (see, e.g., U.S. Pat. Nos. 4,683,195; 4,683,202; and 4,965,188). PCR generally comprises mixing a sample, e.g., a sample comprising a gene of interest, e.g., FcγRIIIa gene, with PCR components such as DNA polymerase, dNTPs, buffer, and oligonucleotides to form a PCR mixture, and subjecting the PCR mixture to at least one cycle comprising the steps of denaturing, annealing (or hybridizing), and elongating (or extending). One skilled in the art will recognize that the denaturing, annealing, and elongating steps of PCR may be effectuated by altering the temperature of the PCR mixture. One of skill in the art will also recognize that the temperatures, the length of time at such temperatures, and the number of PCR cycles that the PCR mixture must be subjected to will differ for different oligonucleotides. Additionally, a skilled artisan will recognize that “hot starts” often begin PCR methods, and that a final incubation at about 68° C. or 72° C. may optionally be added to the end of any PCR reaction.

As disclosed herein, the terms “first round of PCR,” “nested PCR” and the like refer to the initial amplification step, wherein the gene of interest, e.g., the gene to be genotyped, e.g., FcγRIIIa gene, is amplified in a PCR reaction from a sample, e.g., genomic DNA. Genomic DNA can be purchased from a vendor (e.g., Coriell Cell Repositories, Camden, N.J.) or can be isolated from a cell population, e.g., whole blood, e.g., human whole blood.

The gene of interest, e.g., FcγRIIIa, is first amplified in a nested PCR reaction with gene-specific primers, e.g., primers that anneal to the nucleotide sequence of FcγRIIIa but not FcγRIIIb, e.g., a portion of the nucleotide sequence with a significant percentage of mismatch between FcγRIIIa and FcγRIIIb. One skilled in the art will recognize that gene-specific primers are primers that anneal specifically to, e.g., FcγRIIIa under stringent conditions.

Annealing reactions, also referred to as hybridization reactions, can be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 3 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 3

| | Poly- | Hybrid | Hybridization | Wash |
|---|---|---|---|---|
| Stringency | nucleotide | Length | Temperature and | Temperature |
| Condition | Hybrid | (bp)¹ | Buffer² | and Buffer² |
| A | DNA:DNA | >50 | 65° C.; 1X SSC | 65° C.; 0.3X SSC |
| | | | -or- | |
| | | | 42° C.; 1X SSC, | |
| | | | 50% formamide | |
| B | DNA:DNA | <50 | T_{B}*; 1X SSC | T_{B}*; 1X SSC |
| C | DNA:RNA | >50 | 67° C.; 1X SSC | 67° C.; 0.3X SSC |
| | | | -or- | |
| | | | 45° C.; 1X SSC, | |
| | | | 50% formamide | |
| D | DNA:RNA | <50 | T_{D}*; 1X SSC | T_{D}*; 1X SSC |
| E | RNA:RNA | >50 | 70° C.; 1X SSC | 70° C.; 0.3xSSC |
| | | | -or- | |
| | | | 50° C.; 1X SSC, | |
| | | | 50% formamide | |
| F | RNA:RNA | <50 | T_{F}*; 1X SSC | T_{f}*; 1X SSC |
| G | DNA:DNA | >50 | 65° C.; 4X SSC | 65° C.; 1X SSC |
| | | | -or- | |
| | | | 42° C.; 4X SSC, | |
| | | | 50% formamide | |
| H | DNA:DNA | <50 | T_{H}*; 4X SSC | T_{H}*; 4X SSC |
| I | DNA:RNA | >50 | 67° C.; 4X SSC | 67° C.; 1X SSC |
| | | | -or- | |
| | | | 45° C.; 4X SSC, | |
| | | | 50% formamide | |
| J | DNA:RNA | <50 | T_{J}*; 4X SSC | T_{J}*; 4X SSC |
| K | RNA:RNA | >50 | 70° C.; 4X SSC | 67° C.; 1X SSC |
| | | | -or- | |
| | | | 50° C.; 4X SSC, | |
| | | | 50% formamide | |
| L | RNA:RNA | <50 | T_{L}*; 2X SSC | T_{L}*; 2X SSC |
| M | DNA:DNA | >50 | 50° C.; 4X SSC | 50° C.; 2X SSC |
| | | | -or- | |
| | | | 40° C.; 6X SSC, | |
| | | | 50% formamide | |
| N | DNA:DNA | <50 | T_{N}*; 6X SSC | T_{N}*; 6X SSC |
| O | DNA:RNA | >50 | 55° C.; 4X SSC | 55° C.; 2X SSC |
| | | | -or- | |
| | | | 42° C.; 6X SSC, | |
| | | | 50% formamide | |
| P | DNA:RNA | <50 | T_{P}*; 6X SSC | T_{P}*; 6X SSC |
| Q | RNA:RNA | >50 | 60° C.; 4X SSC | 60° C.; 2X SSC |
| | | | -or- | |
| | | | 45° C.; 6X SSC, | |
| | | | 50% formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length,Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1xSSC = 0.165M).
Additional examples of stringency conditions for polynucleotide hybridization are provided in Sambrook et al. (1989) Molecular Cloning: A Laboratory Manual, Chs. 9 & 11, Cold Spring Harbor Laboratory Press, Cold Spring Harbor, NY, and Ausubel et al., Eds. (1995) Current Protocols in Molecular Biology, Sects. 2.10 & 6.3-6.4, John Wiley & Sons, Inc., herein incorporated by reference.

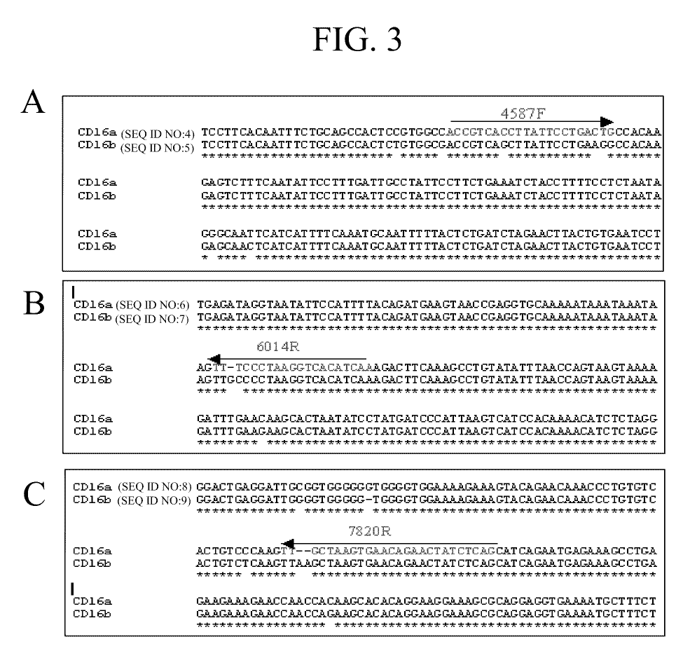

Examples of primers that anneal to the nucleotide sequence of FcγRIIIa but not FcγRIIIb are noted in FIG. 3. For example, nested PCR with 4587F and 7820R primers will result in an amplicon, i.e., FcγRIIIa-specific amplicon.

Preferred primers for pyrosequencing analysis are listed in Table 4. Preferred primers, e.g., primers that anneal to the nucleotide sequence of FcγRIIIa but not FcγRIIIb, may be generated by searching nucleotide sequences against the genomic sequence, e.g., the human genomic sequence, using NCBI BLAST analysis programs. Preferred primers are specific if NCBI BLAST analysis indicates that the PCR primers will match and amplify only the intended target, e.g., the FcγRIIIa, and not other regions in the genome.

TABLE 4

Primers used for the FcγRIIIa 158 F/V Pyrosequencing Analysis

| | | |
|---|---|---|
| Primer Name | Position^{b} | Primer Sequence (5′ to 3′) |

| | | | |
|---|---|---|---|
| 4587F | (SEQ ID NO:10) | 102,080-102,101 | ACCGTCACCTTATTCCTGACTG |
| 7820R | (SEQ ID NO:11) | 98,868-98,893 | CTGAGATAGTTCTGTTCACTTAGCAA |
| CD16aPyroFB | (SEQ ID NO:12) | 101,473-101,499 | AGGCAGGAAGTATTTTCATCATAATTC |
| CD16aPyroR | (SEQ ID NO:13) | 202,290-101,311 | AACTTCCCAGTGTGATTGCAGG |
| CD16aPyroS | (SEQ ID NO:14) | 101,392-101,410 | GACACATTTTTACTCCCAA |

a. biotinylated primer
^{b}position is based on the nucleotide position of the primer relative to the GenBank Accession Number AL590385

One skilled in the art will recognize that a primer(s) that anneals directly 5′ or directly 3′ to the preferred primers of the invention, and primer(s) that overlap by at least one nucleotide with the primer(s) of the invention, may also contain the nucleotide sequence with a significant percentage of mismatch between FcγRIIIa and FcγRIIIb; thus, the primer(s) may specifically amplify the preferred amplicon of the invention, i.e., the FcγRIIIa amplicon. Accordingly, such a primer(s) is encompassed within the scope of the present invention.

“Amplicon” refers to the product of a PCR reaction, e.g., nested PCR reaction, e.g., PCR reaction to amplify a fragment of the FcγRIIIa gene. In one embodiment of the invention, the amplicon is about 1428 base pairs. In a preferred embodiment of the invention, the amplicon is at least 1700 base pairs, preferably about 3234 base pairs. A large amplicon will allow genotyping multiple polymorphic sites.

The terms “polymorphism,” “genetic polymorphism,” “polymorphic site” and the like refer to an occurrence of variable alleles in the same population, which may result in phenotypic difference among members of that population. For example, the FcγRIIIa gene contains a 158 F/V polymorphic site, and the presence of valine at both alleles (V/V) results in more efficient IgGI binding and increased NK cell activation compared to the F/F genotype (Koene et al. (1997) Blood, 90:1109-14; Wu et al., supra).

A genotyping reaction is a reaction(s) that results in determination of the nucleic acid sequence of each allele of the gene of interest. The term “allele” refers to one of two copies of a gene; typically one allele is derived from the mother and one from the father. A number of genotyping reactions are known in the art, including but not limited to, e.g., pyrosequencing reaction, DNA sequencing reaction, MassARRAY MALDI-TOF, RFLP, allele-specific PCR, real-time allelic discrimination, microarray, etc. In a preferred embodiment of the invention, the genotyping reaction comprises the pyrosequencing reaction.

A second round of PCR amplification, e.g., in order to ensure PCR specificity for the gene of interest, can be performed before a genotyping reaction. For instance, the amplicon, e.g., the FcγRIIIa-specific amplicon, can be amplified in a PCR reaction with a pair of primers, e.g., second-round gene-specific primers, wherein one of the pair of second-round gene-specific primers is biotinylated, and wherein the amplification results in a biotinylated amplicon. In one embodiment of the invention, the second-round gene-specific primers for the second round of PCR amplification are CD16aPyroFB and CD16aPyroR primers, wherein the CD16aPyroFB primer is biotinylated. In one embodiment, the second round of PCR amplification results in a biotinylated amplicon comprising about 210 base pairs. Because only one of the pair of second-round gene-specific primers is biotinylated, only one of the two strands of the biotinylated amplicon will be biotinylated.

Following amplification, the biotinylated amplicon can be purified in order to facilitate the pyrosequencing reaction, e.g., by immobilization on streptavidin beads. After the biotinylated amplicon is denatured, in at least one embodiment, the biotinylated strand remains immobilized on streptavidin beads, and is thereby purified. DNA strand denaturation can be performed using the denaturation solution (Biotage, Sweden); however, other methods of DNA denaturation are well known in the art.

Purification of the biotinylated strand of the biotinylated amplicon is followed by pyrosequencing-primer annealing, e.g., CD16aPyroS primer annealing. The pyrosequencing reaction is a sequencing reaction wherein nucleotides are added in a predetermined order based on the known sequence and possible nucleotide variants for the polymorphism, e.g., T or G in the case of FcγRIIIa 158 F/V polymorphism. Pyrophosphate groups are generated upon incorporation of nucleotides into the elongating pyrosequencing primer; and the pyrophosphate is subsequently used in a series of enzymatic reactions to generate ATP. ATP can be used as a cofactor for the luciferase enzyme during the conversion of luciferin into oxyluciferin, resulting in light emission. Thus, in the pyrosequencing reaction, the amount of light generated is proportional to the amount of incorporated nucleotide; and the nucleotide sequence, e.g., the nucleic acid at the polymorphic site, can be determined based on the intensity of emitted light. For example, PyroMark™ software (Biotage, Uppsala, Sweden) generates a graphic representation of the intensity of the emitted light, i.e., a pyrogram, which represents emitted light as peaks, and the intensity of the emitted light is proportional to peak height. Thus, the program can assign the genotype based on the light peak height. One skilled in the art would use the PyroMark™ software based on the manufacturer's instructions. One skilled in the art would also recognize that in the case of the FcγRIIIa gene, as the pyrosequencing primer anneals to the sense strand of the biotinylated amplicon, the T to G substitution that generates the 158 F/V polymorphism, will be read as an A to C substitution.

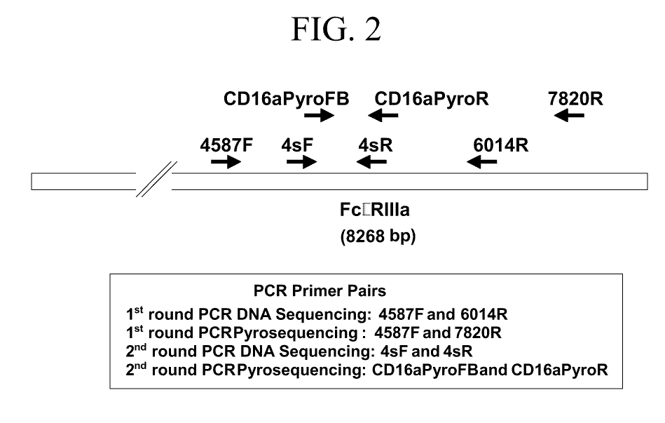

As used herein, DNA sequencing reaction refers to a variation of the dideoxy chain termination DNA sequencing method developed by Fred Sanger, which has been subsequently largely automated. In the methods of the invention, DNA sequencing reaction can be employed for the genotyping reaction, e.g., instead of the pyrosequencing reaction. Alternatively, DNA sequencing reaction can be used as a step in DNA sequencing analysis, wherein the DNA sequencing analysis is used for validation of the accuracy of the pyrosequencing analysis or any other genotyping methods. For example, to confirm genotypes determined using FcγRIIIa 158 F/V pyrosequencing analysis, PCR amplification can be performed to amplify a region of FcγRIIIa gene encompassing the 158 F/V polymorphic site for the purpose of DNA sequencing analysis. The nested PCR can comprise gene-specific primers, e.g., 4587F and 6014R primers. One skilled in the art will recognize that, if DNA sequencing analysis is used for pyrosequencing method validation, it is preferable that different sets of primers are used in DNA sequencing and pyrosequencing analyses. A schematic representation of preferred primers for a first round of PCR (i.e., nested PCR), a second round of PCR, and sequencing for both pyrosequencing and DNA sequencing analyses is depicted in FIG. 2. The use of different PCR strategies is preferred since concordant genotyping results between DNA sequencing analysis and the pyrosequencing analysis provide an additional level of confidence that the pyrosequencing method is specific for the intended target. In a preferred embodiment of the invention, the nested PCR for DNA sequencing analysis results in an amplicon of about 1428 base pairs.

In a preferred embodiment, the second round of PCR for DNA sequencing analysis uses 4sF and 4sR primers (see Treon et al. (2005) J. Clin. Oncol. 23:474-81), and the preferred amplicon is about 245 base pairs. Following the second round of PCR, the PCR product can be purified by methods well known in the art, and sequenced using DNA sequencing reaction. The preferred primer for DNA sequencing reaction of the FcγRIIIa gene is the 146765 primer. Preferred primers for the DNA sequencing analysis are listed in Table 5.

TABLE 5

Primers used for the FcγRIIIa 158 F/V DNA Sequencing
Analysis

| | | |
|---|---|---|
| Primer Name | Position^{a} | Primer Sequence (5′ to 3′) |

| | | | |
|---|---|---|---|
| 4587F | (SEQ ID NO:6) | 102,080-102,101 | ACCGTCACCTTATTCCTGACTG |
| 6014R | (SEQ ID NO:15) | 100,674-100,693 | TTGATGTGACCTTAGGGAA |
| 4Sf | (SEQ ID NO:16) | 101,497-101,522 | GTCACATATTTACAGAATGGCAAAGG |
| 4Sr | (SEQ ID NO:17) | 101,283-101,306 | CCAACTCAACTTCCCAGTGTGATT |
| 146765 | (SEQ ID NO:18) | 101,482-101,499 | AGGCAGGAAGTATTTTCA |

^{a}position is based on the nucleotide position of the primer relative to the GenBank Accession Number AL590385

One skilled in the art will recognize that the method of the present invention can generate genotyping data about several other potentially clinically relevant polymorphisms, e.g., polymorphisms set forth in the NCBI Single Nucleotide Polymorphism database (dbSNP Build 127) as SNP_IDS NOs: 1042223, 1042222, 104222, 375794, 445509, 378618, 448312, 1042215, 1042214, 2499445, 3181668, 7539036, 1042209, 1126552, 1042207, 1042206, 17853189, 10919555, 10800579, 10800580, 10800581, 4657062, 397429, 426615, 10533383, 10624618, 36091086, 449463, 4657063, 370077, 371849, 424288, 3835614, 394678, 449443, 396716, 443082, 5778214, 396991, etc. One skilled in the art will also recognize that the method of the invention may be used to generate genotyping data for any gene of interest. Thus, the methods of the present invention may be used to determine whether a polymorphism is associated with, e.g., a disease condition or abnormality. The methods of the present invention can also be used to assess whether a subject is at risk for, or is afflicted with, a polymorphic disease, i.e., a disease associated with the presence of a polymorphism, e.g., a disease associated with at least one amino acid change. The methods of the invention can also be used, e.g., to determine the course of disease progression, to predict drug efficacy, to design individualized therapy, etc.

Even though the invention has been described with a certain degree of particularity, it is evident that many alternatives, modifications, and variations will be apparent to those skilled in the art in light of the disclosure. Accordingly, it is intended that all such alternatives, modifications, and variations, which fall within the spirit and scope of the invention, be embraced by the defined claims.

The entire contents of all references, patents, and patent applications cited throughout this application are hereby incorporated by reference herein.

### EXAMPLES

The Examples which follow are set forth to aid in the understanding of the invention but are not intended to, and should not be construed to, limit its scope in any way. The Examples do not include detailed descriptions of conventional methods, e.g., PCR steps, PCR reagents, etc. Such methods are well known to those of ordinary skill in the art.

### Example 1

### Pyrosequencing Analysis

### Example 1.1

### Materials and Methods

Genomic DNA was purchased from Coriell Cell Repositories (Camden, N.J.) or isolated from human whole blood. Negative (wild-type, i.e., F/F) and positive (heterozygote polymorphic and/or homozygote polymorphic, i.e., F/V and/or V/V, respectively) genomic DNA control samples (i.e., quality control samples) were included in every pyrosequencing analysis run to evaluate the performance of the method. The genotypes of all quality control samples were verified by DNA sequencing reaction. A “no template control” (containing water instead of genomic DNA) was added in every analytical run to control for potential contamination of the reagents. Genomic DNA control samples that were used in the FcγRIIIa 158 F/V assay were obtained from Coriell Cell Repositories, Camden, N.J., and are listed in Table 6.

TABLE 6
Genomic DNA Control Samples

| Sample Name | FcγRIIIa Genotype^{a} |
|---|---|
| NA17134 | 158 F/F (A/A) |
| NA17228 | 158 V/V (C/C) |
| NA17128 | 158 F/V (A/C) |

^{a}Complementary nucleotides corresponding to genotype are shown in parentheses.

PCR primers were purchased from Eurogentec (San Diego, Calif.). Nested PCR primers were diluted to 5 mM; second round PCR and pyrosequencing reaction primers were diluted to 10 mM. The sequences of primers are listed in Table 4. Each experimental sample, i.e., unknown sample, was subjected to the pyrosequencing analysis in duplicate.

### Example 1.2

### Nested Pyrosequencing PCR

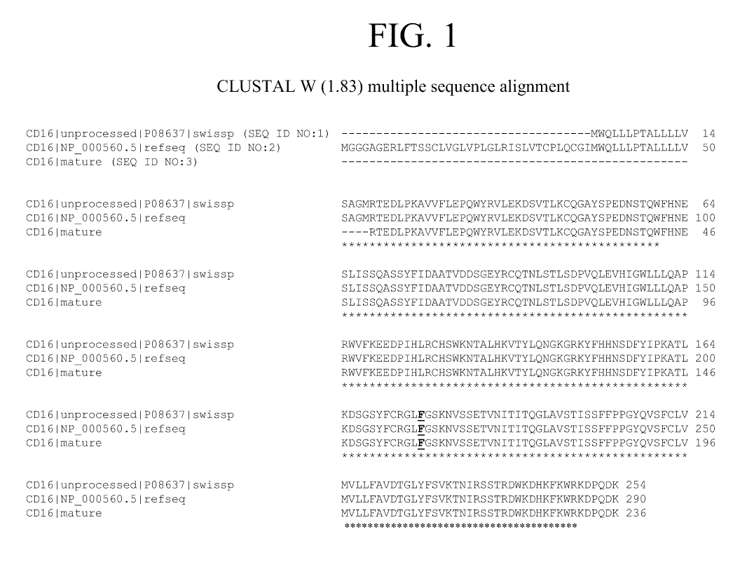

The nested pyrosequencing PCR reaction was performed using the Roche Expand Long Template Kit (Basel, Switzerland). In addition to all the regular PCR components, the nested pyrosequencing PCR reaction used the 4587F and 7820R primers, depicted in FIGS. 1 and 2 and Table 4, and was performed in a 96-well format. The PCR reaction consisted of the steps listed in Table 7.

TABLE 7

| | | |
|---|---|---|
| 94° C. | 2 minutes | 1 cycle |
| 94° C. | 1 minute | 45 |
| 47° C. | 1 minute | |
| 68° C. | 2 minutes and 30 seconds | |
| 68° C. | 7 minutes | 1 cycle |
| 4° C. | HOLD | |

The nested pyrosequencing PCR resulted in an amplicon of 3,234 base pairs and was used as a template for the second round of the pyrosequencing PCR.

### Example 1.3

### Second Round of Pyrosequencing PCR

The second round of pyrosequencing PCR was performed using the Qiagen HotStar Taq Kit (Qiagen, Valencia, Calif.). In addition to all the regular components of the PCR reaction, the second round of pyrosequencing PCR reaction used the CD16aPyroFB and the CD16aR primers, depicted in FIG. 2 and Table 4. The CD16aPyroFB was biotinylated to facilitate subsequent purification of the PCR product. The steps of the second round of the pyrosequencing PCR are listed in Table 8. One microliter of the pyrosequencing nested PCR product was used in the second round of pyrosequencing PCR to amplify a biotinylated amplicon of 210 base pairs. The second round of pyrosequencing PCR resulted in biotinylation of the sense strand of the biotinylated amplicon.

TABLE 8

| | | |
|---|---|---|
| 95° C. | 15 minutes | 1 cycle |
| 95° C. | 20 seconds | 45 |
| 58° C. | 20 seconds | |
| 72° C. | 20 seconds | |
| 72° C. | 5 minutes | 1 cycle |
| 4° C. | HOLD | |

### Example 1.4

### Purification of the Biotinylated Amplicon

The biotinylated PCR product(s), i.e., the biotinylated amplicon, was purified by immobilization on streptavidin-coated sepharose beads (Amersham Biosciences, Uppsala, Sweden). In a 96-well plate containing 5 μL per well of each biotinylated PCR reaction, the volume of the PCR reaction was adjusted to 40 μL with Dnase-free/Rnase-free water (Invitrogen, Carlsbad, Calif.). Two μL of streptavidin sepharose beads per PCR reaction was added to a tube, followed by addition of 40 μL Binding Buffer (Biotage, Uppsala, Sweden) per PCR reaction. The tube was mixed by inverting 4-6 times, and 40 μL of the Binding Buffer-bead mixture was added to each well of the 96-well plate. The reaction was incubated for 5 min at room temperature on a microtiter shaker plate while agitating constantly at 1,400 rpm to keep the beads dispersed.

### Example 1.5

### Biotinylated Strand Separation and Primer Annealing

A plate containing the sequencing primers was prepared so that primer annealing could occur immediately following biotinylated strand separation. The primers were diluted to 0.3 μmol/L using Annealing Buffer (Biotage, Uppsala, Sweden). Twelve μL of diluted CD16aPyroS pyrosequencing primer (depicted in Table 4) was added to each well in a PSQ HS 96 plate (Biotage, Uppsala, Sweden).

Five troughs were placed in the empty spaces on the Vacuum Prep Station (Biotage, Uppsala, Sweden). One trough was filled with 180 mL high purity water, one trough with 180 mL 70% ethanol, one trough with 180 mL washing buffer, and one trough with 120 mL denaturation solution (solutions obtained from Biotage, Uppsala, Sweden).

The probes of the vacuum prep tool (Biotage, Uppsala, Sweden) were primed by lowering the tool into the trough with water for approximately 30 seconds to wash the filter probes. Streptavidin beads, together with the immobilized biotinylated amplicon, were captured on the filter probes by slowly lowering the vacuum prep tool into the PCR plate. The beads were washed by moving and immersing the vacuum prep tool in the trough with 70% ethanol and letting the solution flush through the filters for 5 seconds. The biotinylated strand of the biotinylated amplicon was subsequently separated from the nonbiotinylated strand of the biotinylated amplicon by moving and immersing the prep tool in the trough with denaturation solution and letting the solution flush through the filters for 5 seconds. The final wash was performed by immersing the prep tool in the trough with washing buffer and letting the solution flush through the filters for 5 seconds.

The beads were released by disconnecting the vacuum, and dispensed into a PSQ HS 96-well plate, prefilled with 0.3 μmol/L pyrosequencing primer in 12 μL annealing buffer.

The primer was annealed to the biotinylated strand template by heating the plate with samples at 90° C. for 2 minutes using the PSQ 96 HS Samples Prep Thermoplate Kit and allowing the samples to slowly cool to room temperature.

### Example 1.6

### PyroMark™ Set Up and Pyrosequencing Reaction

In the PyroMark™ set up, Reagent Dispensing Tips (RDTs) dispense enzyme and substrate during the pyrosequencing run. Capillary Dispensing Tips (CDTs) dispense the nucleotides during the pyrosequencing run. All tips were obtained from Biotage. The CDTs and RDTs were washed by filling with water and then applying pressure to the top of the CDT or RDT. The CDTs and RDTs were dried with a light duty tissue wiper, and placed into the Dispensing Tip holder.

The PyroMark™ software indicated the volumes of reagents needed for the run. Using the volumes listed, the appropriate amounts of enzyme and substrate were added to the RDTs, and twice the volume of dATP, dCTP, dGTP and dTTP were added to each CDT. The Dispensing Tip holder and the plate were placed into the PyroMark™ instrument. The pyrosequencing run was completed as per manufacturer's instructions.

The sequence to be analyzed was A/CAAGCCCCCTGCAGAAGTAGGAGCCG (SEQ ID NO:19/20), with the location of the polymorphism indicated by the slash between the two possible nucleotides at the polymorphic position. The dispensation order of the nucleic acids was TCATGCCTGC (SEQ ID NO:21).

Other information on pyrosequencing reactions, PyroMark™ software, etc., is known in the art, and can also be obtained from Biotage (Uppsala, Sweden).

### Example 2

### Pyrosequencing Assay Validation

### Example 2.1

### Assay Specificity

The specificity of the FcγRIIIa 158 F/V pyrosequencing assay was demonstrated by bioinformatics analysis of all PCR primer sequences. Because the FcγRIIIa gene is highly homologous to the FcγRIIIb gene (97% sequence identity), a two-round PCR strategy was employed to ensure specificity for FcγRIIIa—by generating an amplicon in an initial round of PCR, i.e., a first round of PCR, and subsequently using it as the template for a second round of PCR. An alignment of the FcγRIIIa and FcγRIIIb genomic DNA sequences identified a limited number of regions in FcγRIIIa that would be good candidates for first round PCR primer design (FIG. 3). As efficient binding of the 3′ end of a primer is necessary for amplification, primers were designed to maximize the number of mismatches with FcγRIIIb at the 3′ end of the primer. All first round PCR primers were predicted to be specific for FcγRIIIa based on mismatches with the FcγRIIIb gene, as described below.

For instance, relative to FcγRIIIb, the 4587F primer has two mismatches near the 5′ end and a four base pair insertion close to the 3′end. Relative to FcγRIIIb, the 7820R primer has a two base pair insertion very close to the 3′end. Finally, relative to FcγRIIIb, the 6014R primer has a one base pair insertion and one base pair mismatch very close to the 3′end. Pairing of either the 7820R or the 6014R reverse primers with the 4587F primer was predicted by bioinformatics analysis to specifically amplify FcγRIIIa, but not FcγRIIIb.

The specificity of all PCR primers was further analyzed by searching primer sequences against the human genomic sequence using the following BLAST search criteria. Both first round forward and reverse PCR primer sets showed only one perfect hit to the target region of the FcγRIIIa gene. Primer sequences were additionally checked for the possibility of nonspecific amplification, and no single contiguous chromosomal segment in the sequenced human genome (each segment ˜110,000 base pairs in length) was identified as having high homology hits for both forward and reverse primers, indicating that the PCR primer sets would amplify only the targeted region in the FcγRIIIa gene. Based on the results of these in silico analyses, both the sets of primers used for the FcγRIIIa 158 F/V pyrosequencing assay, and for the PCR reaction used to establish the accuracy of pyrosequencing results by DNA sequencing, were predicted to be specific for the FcγRIIIa gene.

### Example 2.2

### Assay Efficiency and Reproducibility

The assay efficiency is defined as the number of samples yielding acceptable genotype calls divided by the total number of samples analyzed, and is expressed as a percentage. The PyroMark™ software generates information regarding the success of the run, indicated as “passed,” “check,” or “failed” scores. The “passed” score indicates successful genotype identification, the “check” score indicates that the pyrosequencing reaction result must be confirmed visually, and the “failed” score indicates a failed pyrosequencing reaction, possibly due to the failed PCR during the first and/or second round(s) of PCR. In order to confirm genotype, either (1) both of the two duplicate runs should have received at least a “check” score, or (2) at least one of the two duplicate runs should have received a “passed” score.

To determine the reproducibility of the present assay, the FcγRIIIa 158 F/V pyrosequencing analysis was performed on three different days using 26 blind-labeled genomic DNA samples (i.e., the results were reported by an analyst who was blinded to the identity of each sample). The samples were then identified, and genotypes reported for each analytical run were compared. The reproducibility was defined as the total number of samples yielding identical genotype calls in all three analytical runs divided by the total number of samples yielding any genotype calls in all three analytical runs.

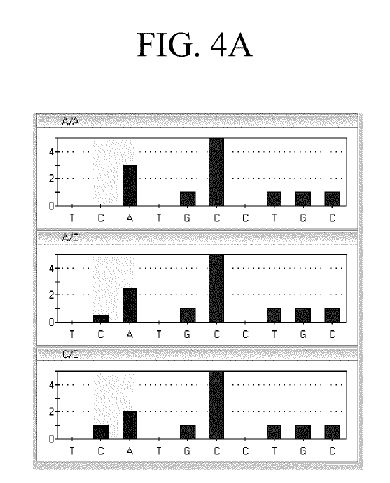

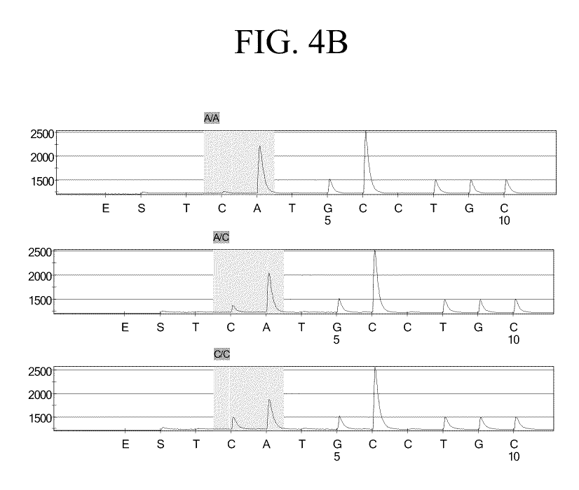

Software-assigned genotype assignments for each sample in each analytical run of each assay are presented in Table 9. The overall assay efficiency was 100% since all 26 validation samples yielded genotype calls in all analytical runs. The reproducibility of the assay was determined to be 100%, as the genotypes for all validation samples were found to be identical on all days. Images of representative theoretical and actual pyrograms for selected validation samples of each possible genotype are shown in FIG. 4A and FIG. 4B, respectively. As expected for each genotype, the pattern of peak intensities at the polymorphic position in the actual pyrogram matched that predicted in the theoretical pyrograms.

TABLE 9
FcΥRIIIa 158 F/V Pyrosequencing Assay
Efficiency and Reproducibility

| Sample ID | Run 1 | Run 2 | Run 3 |
|---|---|---|---|
| 1 | C/C | C/C | C/C |
| 2 | A/A | A/A | A/A |
| 3 | A/C | A/C | A/C |
| 4 | A/A | A/A | A/A |
| 5 | A/A | A/A | A/A |
| 6 | A/A | A/A | A/A |
| 7 | C/C | C/C | C/C |
| 8 | A/C | A/C | A/C |
| 9 | A/C | A/C | A/C |
| 10 | A/C | A/C | A/C |
| 11 | A/A | A/A | A/A |
| 12 | A/C | A/C | A/C |
| 13 | A/C | A/C | A/C |
| 14 | A/A | A/A | A/A |
| 15 | A/C | A/C | A/C |
| 16 | A/A | A/A | A/A |
| 17 | A/A | A/A | A/A |
| 18 | A/C | A/C | A/C |
| 19 | A/C | A/C | A/C |
| 20 | C/C | C/C | C/C |
| 21 | A/C | A/C | A/C |
| 22 | C/C | C/C | C/C |
| 23 | C/C | C/C | C/C |
| 24 | C/C | C/C | C/C |
| 25 | A/A | A/A | A/A |
| 26 | A/A | A/A | A/A |
| Efficiency | 100% | 100% | 100% |

| | |
|---|---|
| Overall Efficiency | 100% |
| Reproducibility | 100% |

### Example 2.3

### Assay Accuracy

To determine the accuracy of the present pyrosequencing assay, all 26 validation samples were submitted to the Wyeth DNA Sequencing Group (Cambridge, Mass.) for an independent genotyping assessment by DNA sequencing analysis (see Examples 2.4-2.7). The overall accuracy of the present pyrosequencing assay was defined as the total number of samples yielding genotype calls identical to those determined by DNA sequencing analysis divided by the total number of samples yielding genotype calls.

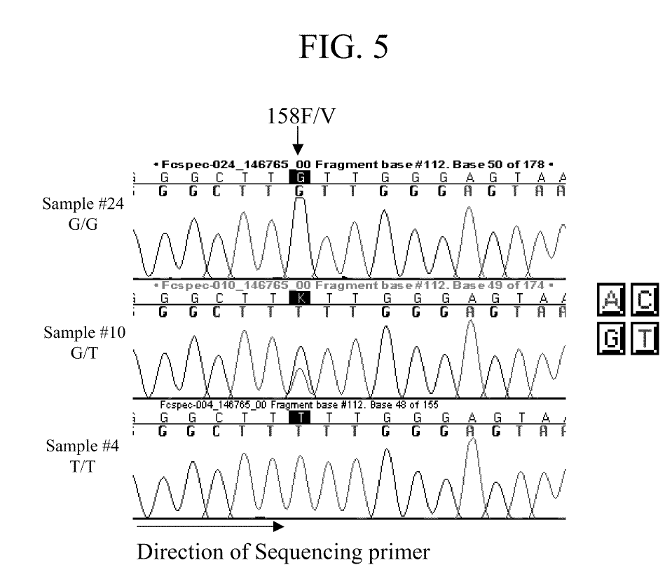

The genotype of each sample determined by DNA sequencing was identical to that determined using the pyrosequencing assay, as shown in Table 10. Representative chromatograms, i.e., graphical representation of DNA sequencing results, for samples of all possible genotypes corresponding to the 158 F/V polymorphism are shown in FIG. 5. For all chromatograms, the nucleotide represented by each peak is indicated at the top; in the case of two peaks a degenerate nucleotide is assigned (i.e., “K”).

TABLE 10
FcΥRIIIa 158 F/V Pyrosequencing Assay Accuracy

| Sample ID | Pyrosequencing Result | DNA Sequencing Result |
|---|---|---|
| 1 | C/C | C/C |
| 2 | A/A | A/A |
| 3 | A/C | A/C |
| 4 | A/A | A/A |
| 5 | A/A | A/A |
| 6 | A/A | A/A |
| 7 | C/C | C/C |
| 8 | A/C | A/C |
| 9 | A/C | A/C |
| 10 | A/C | A/C |
| 11 | A/A | A/A |
| 12 | A/C | A/C |
| 13 | A/C | A/C |
| 14 | A/A | A/A |
| 15 | A/C | A/C |
| 16 | A/A | A/A |
| 17 | A/A | A/A |
| 18 | A/C | A/C |
| 19 | A/C | A/C |
| 20 | C/C | C/C |
| 21 | A/C | A/C |
| 22 | C/C | C/C |
| 23 | C/C | C/C |
| 24 | C/C | C/C |
| 25 | A/A | A/A |
| 26 | A/A | A/A |

| | |
|---|---|
| Accuracy | 100% |

### Example 2.4

### Materials and Methods for DNA Sequencing Analysis

Genomic DNA and primers were obtained as described in Example 1.1. The sequences of primers are listed in Table 4.

### Example 2.5

### Nested DNA Sequencing PCR

The first round of PCR, i.e., nested PCR, for DNA sequencing analysis was performed using Roche Expand Long Template PCR Kit (Roche, Basel, Switzerland). The primers were 4587F and 6014R, and PCR was performed using the steps listed in Table 11.

TABLE 11

| | | |
|---|---|---|
| 94° C. | 2 minutes | 1 cycle |
| 94° C. | 1 minute | 45 |
| 47° C. | 1 minute | |
| 68° C. | 2 minutes and 30 seconds | |
| 68° C. | 7 minutes | 1 cycle |
| 4° C. | HOLD | |

The nested PCR resulted in a 1,428 base pair amplicon, which was used as a template for the second round of PCR.

### Example 2.6

### Second Round of DNA Sequencing PCR

The second round of PCR for DNA sequencing analysis used primers 4sF and 4sR and was performed using the steps listed in Table 12. The second round of PCR amplification resulted in an amplicon of 245 base pairs.

TABLE 12

| | | |
|---|---|---|
| 95° C. | 15 minutes | 1 cycle |
| 95° C. | 20 seconds | 45 |
| 58° C. | 20 seconds | |
| 72° C. | 20 seconds | |
| 72° C. | 5 minutes | 1 cycle |
| 4° C. | HOLD | |

### Example 2.7

### DNA Sequencing Reaction

Following PCR amplification, 5 μL of each reaction was analyzed by agarose gel electrophoresis to confirm that the amplified product was the expected size (i.e., 245 base pairs). The remaining 45 μL of each PCR reaction was purified using the QIAquick PCR Purification kit (Qiagen, Valencia, Calif.) according to the manufacturer's instructions. The concentration of eluted DNA was determined using UV spectrophotometry, and amplicons were sequenced by the Wyeth DNA Sequencing Group (Cambridge, Mass.) using the 146765 primer (listed in Table 5) to sequence the FcγRIIIa polymorphic 158 F/V region.

### Example 3

### Conclusion

A pyrosequencing method for the detection of the 158 F/V polymorphism in the FcγRIIIa gene was investigated and validated. Bioinformatics analysis indicated that the two-round PCR strategy employed in the present assay would amplify only the intended region of the FcγRIIIa gene and would therefore facilitate specific detection of the 158 F/V polymorphism. The assay exhibited 100% efficiency in assigning genotype calls for 26 validation samples across three different days. Under blinded sample conditions, identical genotype determinations were reported for all samples in each analytical run, demonstrating that the assay is reproducible. The accuracy of the method was established by DNA sequencing analysis using an independent PCR-based strategy to amplify FcγRIIIa. The genotypes determined using the FcγRIIIa 158 F/V pyrosequencing assay were in agreement with the results of DNA sequencing for all 26 of the validation samples. The results therefore demonstrate that the FcγRIIIa 158 F/V pyrosequencing assay of the present invention provides specific, efficient, reproducible, and accurate detection of the 158 F/V polymorphism in genomic DNA isolated from human whole blood.

What is claimed is:

1. A method of genotyping at least one polymorphism in a gene of interest, the method comprising:
(a) amplifying the gene of interest in a nested PCR reaction with gene-specific primers to generate a gene of interest-specific amplicon containing at least one polymorphic site; and
(b) performing a genotyping reaction to identify a nucleic acid at the at least one polymorphic site.

2. The method of claim 1, wherein the genotyping reaction is selected from the group consisting of pyrosequencing reaction, DNA sequencing reaction, MassARRAY MALDI-TOF, RFLP, allele-specific PCR, real-time allelic discrimination, and microarray.

3. The method of claim 1, wherein the step of performing a genotyping reaction comprises:
(a) amplifying the gene of interest-specific amplicon in a second round of PCR with second-round gene-specific primers, wherein the amplification results in a biotinylated amplicon, and wherein the biotinylated amplicon comprises one biotinylated strand;
(b) purifying the biotinylated amplicon;
(c) separating the biotinylated strand of the biotinylated amplicon from the nonbiotinylated strand of the biotinylated amplicon;
(d) determining the sequence of the biotinylated strand of the biotinylated amplicon in a pyrosequencing reaction; and
(e) comparing the sequence of the biotinylated strand of the biotinylated amplicon to the known sequence of the gene of interest.

4. The method of claim 1 or 3, wherein the gene of interest is FcγRIIIa.

5. The method of claim 4, wherein the at least one polymorphism is the FcγRIIIa 158 F/V polymorphism.

6. The method of claim 5, wherein the size of the gene of interest-specific amplicon is greater than about 1700 base pairs.

7. The method of claim 6, wherein the size of the gene of interest-specific amplicon is about 3234 base pairs.

8. The method of claim 4, wherein the gene-specific primers are 4587F and 7820R.

9. The method of claim 3, wherein the biotinylated strand is a sense strand.

10. The method of claim 3, wherein the biotinylated strand is an antisense strand.

11. The method of claim 3, wherein the step of purifying the biotinylated amplicon comprises immobilization of the biotinylated amplicon on streptavidin-coated beads.

12. The method of claim 4, wherein the gene-specific primers will anneal to FcγRIIIa but not FcγRIIIb.

13. A method of assessing whether a subject has, or is at risk for, a polymorphic disease comprising detecting at least one polymorphism according to the method of claim 1 or 3.

14. The method of claim 1 or 3, wherein the at least one polymorphic site is selected from the group consisting of polymorphisms identified in the NCBI Single Nucleotide Polymorphism database by SNP_ID NOs: 1042223, 1042222, 104222, 375794, 445509, 378618, 448312, 1042215, 1042214, 2499445, 3181668, 7539036, 1042209, 1126552, 1042207, 1042206, 17853189, 10919555, 10800579, 10800580, 10800581, 4657062, 397429, 426615, 10533383, 10624618, 36091086, 449463, 4657063, 370077, 371849, 424288, 3835614, 394678, 449443, 396716, 443082, 5778214, and 396991.

15. A method of genotyping FcγRIIIa 158 F/V polymorphism, the method comprising:
(a) amplifying FcγRIIIa in a PCR reaction with 4587F and 7820R primers to generate an FcγRIIIa-specific amplicon containing a 158 F/V polymorphic site; and
(b) performing a genotyping reaction to identify a nucleic acid at the 158 F/V polymorphic site on each allele.
